(19) United States
(12) Patent Application Publication
(10) Pub. No.: US 2017/0231814 A1
(43) Pub. Date: Aug. 17, 2017

(54) Comfort Cooling Pad

(71) Applicant: Pamela J. Collins, Annada, MO (US)

(72) Inventor: Pamela J. Collins, Annada, MO (US)

(21) Appl. No.: 15/045,394

(22) Filed: Feb. 17, 2016

(51) Int. Cl. A61F 7/10

(52) U.S. Cl. CPC A61F 7/10; A61F 2007/108

(57) ABSTRACT

Disclosed herein is a cooling pad for use in bringing down the increased temperature of a hyper-thermic patient. The cooling pad could be adapted in the form of a mattress or could be used to cover the body of hyperthermic patient. The cooling pad comprises a flexible and rollable sealed elongated envelope made of multiple layers. The inner layer of envelope is fluid impervious, followed by a protective outer layer. The envelope encloses a fluid having high latent heat of fusion and may further include additives such as viscosity modifying agent.

### CROSS REFERENCE TO RELATED APPLICATIONS

Not applicable

### FEDERALLY SPONSORED RESEARCH AND DEVELOPMENT

Not applicable.

### MICROFICHE

Not applicable

### BACKGROUND OF THE INVENTION

(1) Field of the Invention

The present invention relates to a cooling aid and in particular to a cooling pad for use in bringing down the temperature of hyper-thermic patient.

(2) Background of the Invention

Temperature control is an important function of the body and the hypothalamus is the main temperature-regulating organ in the body. No matter what is the outside temperature, the body temperature is always maintained constant. The hypothalamus takes feedback from heat sensitive receptors and in response to the changes in core temperature of the body, sends nerve impulses to the blood vessels, sweat glands, and muscles to raise or lower the body temperature. The temperature sensitive sensors are mainly located in blood vessels and the skin.

The heat maintaining functions of the body could fail under certain conditions such as a disease or disorder, improper fluid balance, due to certain drugs or extreme weather conditions. An abnormal increase in body temperature is known as hyperthermia and an abnormal decrease in body temperature is known as hypothermia.

Heat stroke is a life threatening form of hyperthermia, and occurs when the body temperature increases significantly. The uncontrolled increase in body temperature possess many health risk to the body and has to be externally controlled by providing cold environment to the body of patient, such that the excess heat could dissipate form the body bringing down the body temperature.

Different methods have been employed to bring down the body temperature, including air-conditioned environment, shower in cold water, application of cold packs etc. When the increased temperature poses risk to the life, the body of the patient has to be covered in ice for immediately bringing down the body temperature. Prior art discloses several apparatuses for providing cold environment to the body, for example, a U.S. Pat. No. 6,764,502 issued to “Arizant Healthcare Inc.” teaching a devices that utilize evaporative, convective, and/or conductive cooling to cool the human body in aid of surgery, medical treatment, therapy, or comfort. Device includes thermal cooling pads or mattresses that are placed beneath a person, and cool by conduction (and optionally evaporation, as well). The pad is wetted with an evaporation liquid, such as water. Wetting may be conducted by hand, or by routing the liquid through an internal liquid flow passage. When an air blower is coupled to the air inlet, air circulates through the airflow passage and evaporative cools the pad by removing warmed water vapor.

Although prior art is rich in cooling apparatuses, the apparatuses of prior arts are however complex, and costly, which limits their use. Thus, a need could be appreciated for a cooling pad that is economic and simple in use. Moreover, the cooling pad could be easily stored and is portable.

### SUMMARY OF THE INVENTION

The present invention, therefore, has as its principal object to provide a cooling pad.

An additional object of the present invention is that the cooling pad is of dimension to cover major portion of an adult human.

Another object of the present invention is that the cooling pad is simple to use.

Yet another object of the present invention is that cooling pad is economic to manufacture.

Certain embodiments of present invention provide an elongated cooling pad configured to lie in spatial relationship with the body of a person permitting transfer of heat from the body to the cooling pad. The cooling pad comprising a flexible and a rollable sealed elongated envelope made of multiple layers. The inner layer of envelope is fluid impervious, and a protective outer layer. The cooling pad is adaptable to lie beneath the body of the patient or cover the top of patient like a blanket. The envelope encloses a fluid having high latent heat of fusion, such as water, glycols, or mixture of water and glycols. The cooling pad may further comprise a viscosity-modifying agent to modify the viscosity of fluid and other additives, such as compounds used to lower the freezing point of liquid.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is the perspective view of the cooling pad lying on the bed.

FIG. 2 is the perspective view of the cooling pad.

### DETAILED DESCRIPTION OF THE INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural and logical changes may be made without departing from the spirit and scope of the present invention. The following detailed description is, therefore, not to be taken in a limiting sense, and the scope of the present invention is defined by the appended claims and their equivalents.

The present invention provides a cooling pad for use in bringing down the temperature of a hyper-thermic patient.

Before explaining at least one embodiment of the invention in detail, it is to be understood that the invention is not limited in its application to the details of construction and the arrangements of the components set forth in the following description. The invention is capable of other embodiments and of being practiced and carried out in various ways. In addition, it is to be understood that the phraseology and terminology employed herein are for the purpose of description and should not be regarded as limiting.

As used in this specification and the appended claims, the singular forms “a”, “an”, and “the” include plural referents unless the content clearly dictates otherwise.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

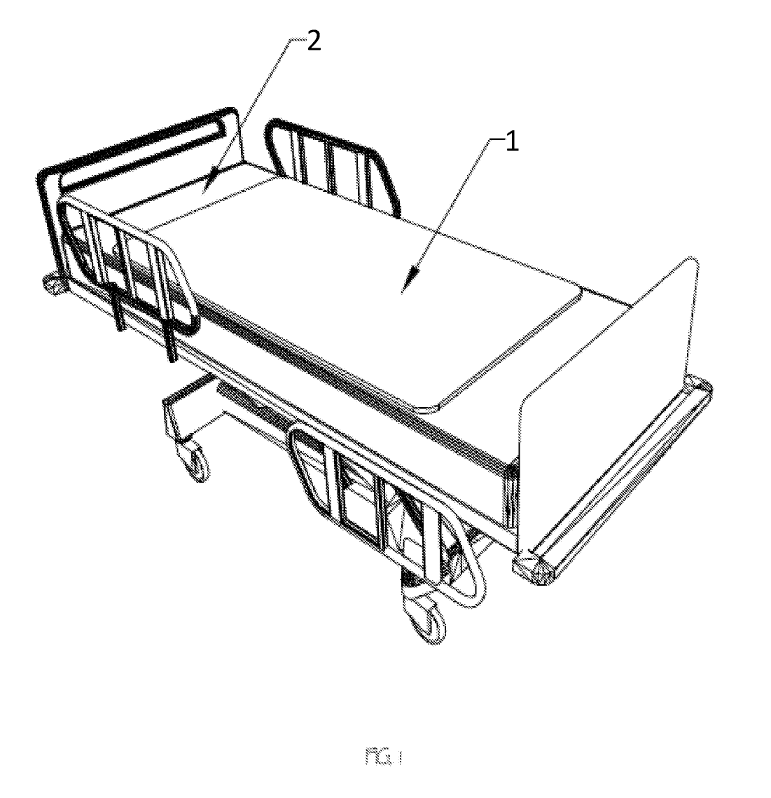

Now, referring to FIG. 1, which shows the top surface of the cooling pad 1 lying on the bed 2. The cooling pad 1 is a rectangular elongated envelope or bag having a top surface and a bottom surface. The envelope is made of flexible material, which allows the envelope to be rolled, and the material should be heat conductive. Practically, the envelope could be made of one or more layers of different material, such as the inner layer of the envelope could be made of fluid impervious material, such as plastic polymers and an outer layer of protective material. The protective layer could be made of fiber, or plastic polymer, which provides flexibility and strength to the cooling pad 1. Moreover, the outer layer allows the cooling pad 1 to be washable and comfortable to the skin of the human. The pad 1 may further includes middle layers or the outer layer is modified such as the skin of the person in contact with the cooling pad 1 is not exposed to very cold temperature, that may harm or do injury to the person/user. For example, the thickness of fiber could be increased to prevent burning of skin from ice-cold water contained in the cooling pad.

The cooling pad 1 contains a fluid, which functions as a carrier of heat. The fluid could be any liquid or semi-solid material having good heat capacity or latent heat of fusion. Preferably, water could be used, as water having excellent latent heat of fusion; moreover, the use of water is safe and environmental friendly. Although, the envelope is sealed to prevent leakage of contained fluid, however, fluid could leak, in case the envelope is damaged. The use of water would not pose any hazard on leakage. Instead of water, other liquids like glycols including propylene glycol or ethylene glycols could be used, or the glycols in combination with water could also be used. The viscosity of the fluid could be modified using viscosity modifying agents such as cellulosic fibers, silica gel or coated silica gel. Further, other additive could be added such as anti-microbial compounds to inhibit microbial growth, compounds used to lower the freezing point of water, and such viscosity modifying agents, antimicrobial compounds and freezing point lowering substances are obvious to a skilled person. The fluid should not freeze when cooled in the refrigerator as it may cause the cooling pad 1 to be rigid.

The cooling pad 1 is activated by cooling the cooling pad 1 in a refrigerator or any other cooling apparatus. The temperature of the cooling should not be below the freezing point of the fluid. The cooling pads 1 could be rolled or folder for storing in the refrigerators and could be taken out and unfolded for use. The cooling pad 1 could be either placed beneath the body of the person, or placed on top of the person like a blanket or could be wrapped around the body.

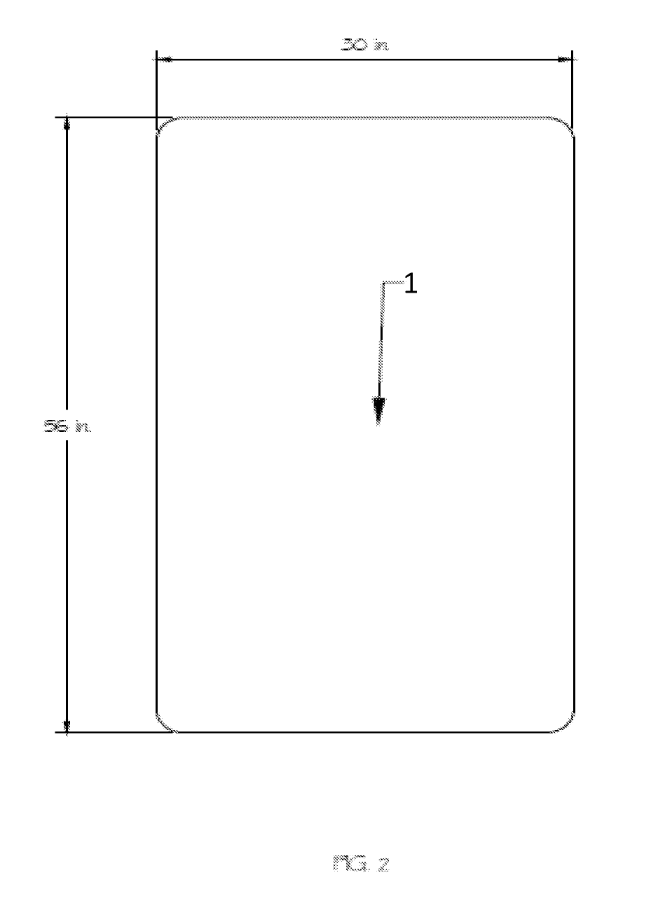

It is obvious that the size of the cooling pad 1 could be varied according to the size of the adult body; preferably, the size of the cooling pad could extend from the shoulder to the ankle of the body. More preferably, the size of the cooling pad 1 is approximately 50 in. in length and 30 in. in width as shown in FIG. 2.

The cooling pad 1 could be further enclosed in a washable or disposable cover. The cover could be a cloth envelope, which could completely enclose the cooling pad 1. The cover could be removed and washed, such that the cover could be again used to enclose the envelope. In addition, the cover could be made of paper material for providing disposable covers. The use of disposable covers could be advantageous in clinics or hospitals wherein the same cooling pad 1 could be frequently used.

Thus, the present invention is advantageous by provides a cooling pad 1 that is simple to use and could be used for hyperthermic persons, i.e. persons having the body temperature higher than normal body temperatures. The invention could be used for person having fever, heat stroke and like wherein the cooling pad could help to lower the body temperature to normal or near normal body temperature. The cooling pad 1 could be used to provide comfort in hot weathers for providing comfort to the user.

It is to be understood that the above description is intended to be illustrative, and not restrictive. For example, the above-discussed embodiments may be used in combination with each other. Many other embodiments will be apparent to those of skill in the art upon reviewing the above description.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

What is claimed is:

1. An elongated cooling pad configured to lie in spatial relationship with body of a person permitting transfer of heat from body of the person to the cooling pad, said cooling pad comprising:
a. a sealed envelope having an inner layer of fluid impervious material, and an outer protective layer; and
b. a fluid enclosed in said sealed envelope, the fluid having a high latent heat of fusion.

2. The elongated cooling pad according to claim 1, wherein said fluid is water.

3. The elongated cooling pad according to claim 1, wherein said elongated cooling pad further comprises a viscosity modifying agent.

4. The elongated cooling pad according to claim 1, wherein said outer protective layer of the elongated cooling pad is washable.

5. The elongated cooling pad according to claim 1, wherein said cooling pad lie beneath the body of the patient.

6. The elongated cooling pad according to claim 1, wherein said cooling pad covers the body of the person.

7. The elongated cooling pad according to claim 1, wherein said cooling pad is flexible and could be rolled to store in a refrigerator, said rolled cooling pad could be unrolled after cooling in the refrigerator.

8. The elongated cooling pad according to claim 1, wherein said person is suffering from hyperthermia, said cooling pad when in contact with skin of the person brings down the body temperature of the person.

9. The elongated cooling pad according to claim 1, wherein said person is suffering from heat stroke, said cooling pad when in contact with skin of the person brings down the body temperature of the person.

10. The elongated cooling pad according to claim 1, wherein said person is suffering from fever, said cooling pad when in contact with skin of the person brings down the body temperature of the person.

11. The elongated cooling pad according to claim 1, wherein said elongated cooling pad further comprises a removable outer cover, said cover configured to completely enclose said cooling pad, said cover made of disposable paper or fiber material.

12. A method of decreasing temperature of the body of a hyper-thermic person using a cooling pad, wherein said cooling pad comprises:
a. a sealed envelope having an inner layer of fluid impervious material, and an outer protective layer; and
a. a fluid enclosed in said sealed envelope, the fluid having a high latent heat of fusion;
said cooling pad configured to lie in spatial relationship with body of the hyper-thermic person permitting transfer of heat from body of the hyper-thermic person to the cooling pad.

13. The method according to claim 12, wherein said cooling pad is placed beneath the body of hyper-thermic person.

14. The method according to claim 12, wherein said cooling pad is covered on the front body portion of the hyper-thermic person.

15. The method according to claim 12, wherein said fluid is water.

16. The method according to claim 12, wherein said cooling pad further comprises a viscosity modifying agent.

17. The method according to claim 12, wherein said outer protective layer of the cooling pad is washable.

18. The method according to claim 12, wherein said hyper-thermic patient is suffering from fever.

19. The method according to claim 12, wherein said hyper-thermic patient is suffering from heat stroke.

20. The method according to claim 12, wherein said cooling pad is further comprises a removable outer cover, said cover configured to completely enclose said cooling pad, said cover made of disposable paper or fiber material.
